(19) United States
(12) Patent Application Publication
(10) Pub. No.: US 2023/0221389 A1
(43) Pub. Date: Jul. 13, 2023

(54) METHOD FOR PERFORMING A MAGNETIC RESONANCE MEASUREMENT, A MAGNETIC RESONANCE APPARATUS, AND A COMPUTER PROGRAM PRODUCT

(71) Applicant: Siemens Healthcare GmbH, Erlangen (DE)

(72) Inventors: Stephan Biber, Erlangen (DE); Peter Speier, Erlangen (DE)

(21) Appl. No.: 18/096,558

(22) Filed: Jan. 12, 2023

(30) Foreign Application Priority Data
Jan. 13, 2022 (EP) 22151380.7

(51) Int. Cl. G01R 33/54; G01R 33/28; A61B 5/055

(52) U.S. Cl. CPC G01R 33/546; G01R 33/283; A61B 5/055

(57) ABSTRACT

A method for performing a magnetic resonance measurement includes selecting a first set of coil elements from a plurality of coil elements and a second set of coil elements from the plurality of coil elements, and performing a magnetic resonance measurement. During the magnetic resonance measurement with the first set of coil elements and the second set of coil elements, magnetic resonance signals and pilot tone signals are received. The method includes ascertaining at least one magnetic resonance image solely with the assistance of magnetic resonance signals received with the first set of coil elements during performance of the magnetic resonance measurement, and ascertaining patient movement information solely with the assistance of pilot tone signals received with the second set of coil elements during performance of the magnetic resonance measurement. The first set of coil elements is not congruent with the second set of coil elements.

This application claims the benefit of European Patent Application No. EP 22151380.7, filed on Jan. 13, 2022, which is hereby incorporated by reference in its entirety.

### BACKGROUND

The present embodiments relate to a method for performing a magnetic resonance measurement, a magnetic resonance apparatus, and a computer program product.

In medical technology, imaging by magnetic resonance (MR), also known as magnetic resonance tomography (MRT) or Magnetic Resonance Imaging (MRI), is distinguished by high soft tissue contrast. When performing a magnetic resonance measurement of a patient, a magnetic resonance apparatus is used to irradiate radiofrequency (RF) pulses for generating an RF field and gradient pulses for generating magnetic field gradients into an examination region in which the patient is situated. This triggers spatially encoded magnetic resonance signals in the patient.

The magnetic resonance signals are received by the magnetic resonance apparatus and used to reconstruct magnetic resonance images. This magnetic resonance signal may be received by local receiving coils, or “local coils”, that are arranged in the immediate vicinity of the patient in order to achieve a better signal-to-noise ratio (SNR). The local coils conventionally have one or more coil elements that are configured to receive RF signals (e.g., magnetic resonance signals).

Documents U.S. Pat. No. 10,222,443 B2 and Speier et al. PT-Nav: A Novel Respiratory Navigation Method for Continuous Acquisition Based on Modulation of a Pilot Tone in the MR-Receiver. ESMRMB, 129:97-98, 2015 disclose a method that makes it possible to trigger time sequences during magnetic resonance measurement of physiological movements, such as, for example, respiration and/or heartbeat. In this way, movement artifacts may be avoided (e.g., prospective movement correction) and/or eliminated in the course of digital post-processing (e.g., retrospective movement correction).

This conventionally involves a conventionally small pilot tone signal generator emitting a weak RF signal that is sufficiently constant (e.g., with regard to amplitude and/or frequency). Such a pilot tone signal generator is described in document U.S. Pat. No. 10,393,845 B2, for example. The emitted signal interacts with the patient and is then received as a pilot tone signal by coil elements of a receive coil, conventionally coil elements of the local coils. Such a receive coil may also be configured to transmit RF signals (e.g., the coil may also be a transmit/receive coil). As already described above, the receive coils are conventionally used at the same time for imaging (e.g., for receiving magnetic resonance signals). Spectral separation of the pilot tone signal from the magnetic resonance signal in the received RF signal allows perturbation-free capture of image-forming magnetic resonance signals and simultaneous reception of the pilot tone signal. The amplitude and phase of the pilot tone signal may then be used to capture the time profile of physiological signals, such as respiration and/or heartbeat. The magnetic resonance signals of the received RF signal are used to reconstruct magnetic resonance images.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an improved method for performing magnetic resonance measurement of a patient is provided. For example, still more targeted use of the pilot tone technique may be provided.

In one embodiment, a method (e.g., a computer-implemented method) for performing magnetic resonance measurement of a patient using a magnetic resonance apparatus is provided. The magnetic resonance apparatus includes, for example, a plurality of coil elements for receiving RF signals (e.g., magnetic resonance signals and/or pilot tone signals). A first set of coil elements is selected from the plurality of coil elements (e.g., in order to receive magnetic resonance signals). The first set of coil elements includes at least one first coil element. Further, a second set of coil elements is selected from the plurality of coil elements (e.g., in order to receive pilot tone signals). The second set of coil elements includes at least one second coil element. In this case, the first set of coil elements is not congruent with the second set of coil elements. However, the first set of coil elements may overlap with the second set of coil elements. Further, magnetic resonance measurement is performed, where, during the magnetic resonance measurement with the first set of coil elements and the second set of coil elements, magnetic resonance signals and pilot tone signals are received.

Further, at least one magnetic resonance image is ascertained solely with the assistance of magnetic resonance signals received with the first set of coil elements during performance of the magnetic resonance measurement. Further, patient movement information (e.g., physiological information) is ascertained solely with the assistance of pilot tone signals received with the second set of coil elements during performance of the magnetic resonance measurement.

Through the targeted selection of coil elements from the plurality of coil elements, in order to ascertain one or more magnetic resonance images from the RF signals (e.g., magnetic resonance signals) received with these coil elements, and through the targeted selection of coil elements from the plurality of coil elements, in order to ascertain movement information from the RF signals (e.g., pilot tone signals) received with these coil elements, the pilot tone technique may be made applicable beyond the previous field of application thereof.

For example, the ascertained at least one magnetic resonance image and the ascertained movement information relate to different spatial regions of the patient (e.g., of the patient's body). In one embodiment, the first set of coil elements is selected with regard to suitability for receiving magnetic resonance signals in order to ascertain the at least one magnetic resonance image. In one embodiment, the second set of coil elements is selected with regard to their suitability for receiving pilot tone signals in order to ascertain the movement information. Through targeted selection of the respective coil elements, both purposes may be better accommodated.

Dedicated selection of the coil elements is advantageous, for example, if the imaging volume (e.g., the spatial region from which the at least one magnetic resonance image is ascertained) from a spatial region in which a movement of interest (e.g., a physiological process) of the patient takes place. This would be the case, for example, during measurement of a patient's head with heartbeat and/or respiratory synchronization. For example, targeted selection of the coil elements with regard to purpose advantageously avoids image artifacts that arise through signals flowing into the reconstruction of the at least one magnetic resonance image that originate from regions, for example, far outside the imaging volume.

Instead of acquiring a movement (e.g., physiological movement) in the imaging volume, the movement may be measured at another location where the movement is more readily detectable. This may be demonstrated using the example of a head investigation: in large vessels, blood flow is conventionally pulsatile. When it comes to capturing magnetic resonance signals, synchronization with the flow status of the blood may be provided. The flow is produced by the heart's pumping. Therefore, by observing the movement of the heart with the pilot tone signals, this synchronization may be achieved since the bloodstream links the movement detected by the pilot tone signal with a movement in the imaging volume.

Another example of application are magnetic resonance measurements in which a frequency of the magnetic resonance in the head changes as a function of respiratory movement. In this respect, use may be made of the fact that respiratory movement detected by the pilot tone signal directly changes the resonant frequency in the surroundings (e.g., also in the head).

Further, the ascertained movement information may be used (e.g., by an operator of the magnetic resonance apparatus), for example, to monitor the state, for example, of the patient during magnetic resonance measurement. For example, a conclusion may be drawn as to the state of the patient (e.g., whether the patient is nervous or asleep) based on the movement information (e.g., respiration). The method of the present embodiments may thus efficiently, for example, also enable examinations in which the physiological parameters are not used for the MR capture per se (e.g., in the form of a movement correction of the magnetic resonance signals), but rather, for example, to identify exactly how the patient is feeling.

It is advantageous for respiratory monitoring to detect pilot tone signals from the abdominal region. If, for example, magnetic resonance images of the patient's head or knee are ascertained, the imaging volume is relatively far from the abdomen. By using in each case a separate set of coil elements (e.g., thus the first set and the second set, where these sets may overlap), both optimal imaging and optimal monitoring of physiological parameters may be achieved.

In one embodiment, the method of the present embodiments is used for examinations in which respiratory or heartbeat synchronization contributes to stabilizing the result or makes the measurement possible in the first place, despite the rib cage not being situated in the imaging volume. Such examinations include, for example, functional magnetic resonance imaging (fMRI) of the patient's head or non-contrast magnetic resonance angiography such as, for example, Quiescent Inflow Single Shot (QISS) or Non-contrast MRA of ArTerIes and VEins (NATIVE) in the legs.

To generate pilot tone signals, a pilot tone signal generator may be used to generate RF signals that interact with the patient. A coil element receiving the pilot tone signal may, for example, be part of a coil (e.g., a local coil and/or a body coil fixedly installed in the magnetic resonance apparatus). A coil element receiving the pilot tone signal may be configured to transmit the received pilot tone signal for ascertaining the patient movement information (e.g., for evaluating information about a physiological process in the patient and/or a movement of the patient) to a system control unit of the magnetic resonance apparatus.

The pilot tone signal generator may, for example, be part of a local coil (e.g., integrated and/or installed in a local coil). In one embodiment, the pilot tone signal generator may be positioned particularly close to the patient, such that the pilot tone signals received by the coil elements may have a particularly high SNR.

The RF signal generated by the pilot tone signal generator may have a first frequency band, where the receiving coil element is configured to capture a receive frequency band that includes the first frequency band. The magnetic resonance apparatus may include a radio-frequency antenna unit that is configured to output an RF pulse with a second frequency band. The receiving coil elements may be configured to receive a magnetic resonance signal of the RF pulse, where the magnetic resonance signal has a third frequency band that lies at least substantially outside the first frequency band. In one embodiment, the first frequency band does not collide with the actual measurement signal, the magnetic resonance signal, which lies in the third frequency band.

Selection of the first set of coil elements from the plurality of coil elements and/or selection of the second set of coil elements from the plurality of coil elements may proceed, for example, by a system control unit of the magnetic resonance apparatus. The system control unit may, for example, include one or more processors and/or one or more storage modules.

The first set of coil elements and the second set of coil elements may form a union. The number of elements (e.g., coil elements) in this union may be less than or equal to a maximum available number of receive channels of the magnetic resonance apparatus, where each of the receive channels is in each case configured to receive an RF signal received from a coil element and, for example, to forward the RF signal to a system control unit of the magnetic resonance apparatus. The received RF signal may, for example, include a magnetic resonance signal and/or a pilot tone signal.

The magnetic resonance apparatus, for example, has 16 coil channels; the first set of coil elements has ten coil elements; the second set of coil elements has eight coil elements; two coil elements are both part of the first set of coil elements and the second set of coil elements (e.g., at least one magnetic resonance image and one item of patient movement information is also ascertained using the magnetic resonance signals and pilot tone signals received by these two coil elements); the union thus includes 16 coil elements; each of the 16 coil elements may be read out in each case with one receive channel of the magnetic resonance apparatus.

Performance of the magnetic resonance measurement may, for example, include emission of RF pulses and gradient pulses according to a specified magnetic resonance sequence. The RF pulses may, for example, include excitation pulses and/or refocusing pulses. The gradient pulses may, for example, include slice selection gradient pulses, phase encoding gradient pulses, and/or read-out gradient pulses.

Ascertaining at least one magnetic resonance image may, for example, include a reconstruction of the magnetic resonance signals that was received (e.g., solely) by the coil elements of the first set of coil elements Ascertaining patient movement information may, for example, include the evaluation of the pilot tone signals that was received (e.g., solely) by the coil elements of the second set of coil elements.

The fact that the first set of coil elements is not congruent with the second set of coil elements may, for example, provide that a first part of the union of the first set of coil elements and the second set of coil elements are coil elements that belong only to the first set of coil elements, that a second part of the union of the first set of coil elements and the second set of coil elements are coil elements that belong only to the second set of coil elements, and that a third part of the union of the first set of coil elements and the second set of coil elements are coil elements that belong to both the first and second sets of coil elements. The third part of the union thus constitutes the intersection of the first set of coil elements and the second set of coil elements. In one embodiment, the intersection may be an empty set (e.g., one that does not contain any coil elements having received RF signals that are used both to ascertain the at least one magnetic resonance image and to ascertain the movement information).

The cardinality (e.g., the number of elements of the respective set) of the first set of coil elements and the second set of coil elements may amount to at least one (e.g., both the first set of coil elements and the second set of coil elements may include at least one coil element).

Selection of the first set of coil elements (e.g., for receiving magnetic resonance signals) may proceed prior to selection of the second set of coil elements (e.g., for receiving pilot tone signals). The first set of coil elements may be selected first, and then, the second set of coil elements is selected. Selection of the second set of coil elements may proceed as a function of the first set of coil elements. The first set of coil elements may be selected with a higher priority than the second set of coil elements. For example, the coil elements that are particularly suitable for imaging may be selected first, followed by the coil elements that are particularly suitable for detecting the movement signal. This selection sequence may be advantageous, for example, when the quality of the at least one magnetic resonance image is particularly important compared with the quality of the movement information.

For example, the cardinality of the first set of coil elements and a maximum number of receiving channels of the magnetic resonance apparatus defines a maximum number of coil elements that belong to the second set of coil elements, but not to the first set of coil elements. If the magnetic resonance apparatus includes M receiving channels, for example, and the first set of coil elements has N coil elements, the second set of coil elements may have a maximum of L=M−N coil elements that do not belong to the first set of coil elements. In one embodiment, the cardinality of the second set of coil elements may amount to up to M (e.g., for as many as all the coil elements from the first set of coil elements also to belong to the second set of coil elements).

In one embodiment, selection of the second set of coil elements (e.g., for receiving pilot tone signals) may proceed before selection of the first set of coil elements (e.g., for receiving magnetic resonance signals). The second set of coil elements may be selected first, and then, the first set of coil elements is selected. Selection of the first set of coil elements may proceed as a function of the second set of coil elements. The second set of coil elements may be selected with a higher priority than the first set of coil elements. For example, the coil elements that are particularly suitable for detecting the movement signal may be selected first, followed by the coil elements that are particularly suitable for imaging. This selection sequence may be advantageous, for example, when the quality of the movement information is particularly important compared with the quality of the at least one magnetic resonance image.

For example, the cardinality of the second set of coil elements and a maximum number of receiving channels of the magnetic resonance apparatus defines a maximum number of coil elements that belong to the first set of coil elements, but not to the second set of coil elements. If the magnetic resonance apparatus includes M receiving channels, for example, and the second set of coil elements has L coil elements, the first set of coil elements may have a maximum of N=M−L coil elements that do not belong to the second set of coil elements. In one embodiment, the cardinality of the first set of coil elements may amount to up to M (e.g., for as many as all the coil elements from the second set of coil elements also to belong to the first set of coil elements).

In one embodiment, the first set of coil elements (e.g., for receiving magnetic resonance signals) and the second set of coil elements (e.g., for receiving pilot tone signals) may be jointly selected. The first set of coil elements may be selected parallel to the second set of coil elements. Selection of the first set of coil elements and the second set of coil elements proceeds in mutually dependent manner. The first set of coil elements may be selected with the same priority as the second set of coil elements. For example, the coil elements that are particularly suitable for imaging and the coil elements that are particularly suitable for detecting the movement signal are jointly selected. This selection sequence may be, for example, when the quality of the at least one magnetic resonance image is of similar importance to the quality of the movement information.

A weighting factor that describes a relative prioritization of a probable quality of the at least one magnetic resonance image to be ascertained compared with the quality of the movement information to be ascertained may be defined. With the assistance of this weighting factor, joint selection of the first set of coil elements and the second set of coil elements may proceed, for example, fully automatically and/or semi-automatically.

For example, the number of receiving channels of the magnetic resonance apparatus defines a maximum cardinality of the union of the first set of coil elements and the second set of coil elements. For example, the number of different coil elements that belong to the first set of coil elements and to the second set of coil elements is less than or equal to the number of receiving channels of the magnetic resonance apparatus.

In one embodiment, at least for a part of the plurality of coil elements of the magnetic resonance apparatus, the position thereof relative to the magnetic resonance apparatus and/or relative to the patient is ascertained. Selection of the first set of coil elements (e.g., for receiving magnetic resonance signals) and/or the second set of coil elements (e.g., for receiving pilot tone signals) in each case proceeds, for example, as a function of the respectively ascertained position.

In one embodiment, when the position of the coil elements is known, it is more readily possible to estimate whether or to what extent the respective coil element is suitable for ascertaining magnetic resonance signals for ascertaining the at least one magnetic resonance image and/or pilot tone signals for ascertaining patient movement information.

If a coil element is located in the vicinity of the patient's heart, for example, particularly good pilot tone signals for ascertaining the patient's heartbeat may be received. If a coil element is located in the vicinity of the patient's abdomen, for example, particularly good pilot tone signals (e.g., such signals with a high SNR) for ascertaining the patient's respiratory movement may be received.

Selection of the first set of coil elements (e.g., for receiving magnetic resonance signals) and/or the second set of coil elements (e.g., for receiving pilot tone signals) may proceed, moreover, as a function of a magnetic resonance measurement type or technique and/or of a region of the patient to be examined.

If the region of the patient to be examined is, for example, the patient's head and a coil element is located, for example, in the vicinity of the patient's head, the coil element may receive particularly good magnetic resonance signals (e.g., with a high SNR) for ascertaining the at least one magnetic resonance image.

If the magnetic resonance measurement type or technique is an fMRT or a non-contrast magnetic resonance angiography, for example, the patient's heartbeat and/or respiratory movement may be ascertained. Coil elements that are subsequently positioned by the patient's heart and/or on his/her abdomen may therefore be selected.

The positions of the coil elements may be ascertained by evaluating stored coil-specific information that describes a position of at least one coil element relative to the receive coil including the at least one coil element, and/or by evaluating magnetic resonance signals that were received during an adjustment measurement with the coil elements, and/or by evaluating a video signal that was captured with at least one camera, and/or by evaluating a sensor signal that was acquired with at least one position sensor (e.g., Hall signal/RFID sensor).

The stored coil-specific information may, for example, include a relative position (e.g., in the form of coordinates or an item of information, respectively “internally” or “externally”) of a coil element within the receive coil.

The adjustment measurement may, for example, be performed prior to the magnetic resonance measurement. In one embodiment, such an adjustment measurement lasts only a short time (e.g., just a few seconds). During the evaluation of magnetic resonance signals of the adjustment measurement to ascertain the positions of the coil elements, amplitudes and/or phases of the magnetic resonance signals may be evaluated.

The camera for capturing the video signal may, for example, include a 2D camera and/or a 3D camera. The sensor for detecting the sensor signal may, for example, include a Hall sensor and/or an RFID sensor.

A position of a receive coil relative to the patient may be ascertained using the video signal and/or the sensor signal. In one embodiment, the positions of the coil elements of the receive coils relative to the patient may be ascertained using the position of a receive coil relative to the patient in combination with the stored coil-specific information.

Selection of the first set of coil elements (e.g., for receiving magnetic resonance signals) and/or of the second set of coil elements (e.g., for receiving pilot tone signals) may take place fully automatically and/or semi-automatically.

For example, selection of the first set of coil elements and the second set of coil elements proceeds fully automatically. For example, selection of the first set of coil elements proceeds fully automatically, and selection of the second set of coil elements proceeds fully automatically. For example, selection of the first set of coil elements proceeds fully automatically, and selection of the second set of coil elements proceeds semi-automatically. For example, selection of the first set of coil elements proceeds semi-automatically, and selection of the second set of coil elements proceeds semi-automatically. For example, selection of the first set of coil elements proceeds manually, and selection of the second set of coil elements proceeds fully automatically. For example, selection of the first set of coil elements proceeds manually, and selection of the second set of coil elements proceeds semi-automatically. For example, selection of the first set of coil elements proceeds manually, and selection of the second set of coil elements proceeds manually. For example, selection of the first set of coil elements proceeds fully automatically, and selection of the second set of coil elements proceeds manually. For example, selection of the first set of coil elements proceeds semi-automatically, and selection of the second set of coil elements proceeds manually.

In the case of fully automatic selection of coil elements, an operator of the magnetic resonance apparatus may play no part in selection of the coil elements. Fully automatic selection may be performed solely by the system control unit of the magnetic resonance apparatus. Fully automatic selection may be performed very rapidly and/or very conveniently.

In the case of semi-automatic selection, a set of coil elements may be automatically proposed to the operator, and the operator selects the coil elements (e.g., manually) based on this proposal. Semi-automatic selection may be performed rapidly, conveniently, and/or particularly safely, since the operator has an opportunity to intervene.

In the case of manual selection of the coil elements, the available coil elements that the operator may select or deselect (e.g., manually) may, for example, be indicated to the operator on a screen. In one embodiment, manual selection may be performed flexibly and/or simply implemented.

Selection (e.g., fully automatic selection and/or the automatic proposal provided in the case of semi-automatic selection) of the second set of coil elements proceeds with the assistance of one type of at least one receive coil, which includes at least one part of the plurality of coil elements of the magnetic resonance apparatus, and/or a relative position of the coil elements within at least one receive coil, which includes at least one part of the plurality of coil elements of the magnetic resonance apparatus, and/or at least one characteristic of the patient (e.g., height, weight, sex, and/or a positioning of the patient in the magnetic resonance apparatus, such as on the patient couch).

The type of the at least one receive coil may be characterized in that the receive coil is configured to measure a given region of the patient's body (e.g., a spinal column coil and/or a flexible local receive coil that may be particularly readily placed on the patient's abdomen) and/or has a pilot tone signal generator.

The coil elements to be selected for the second set of coil elements may lie as far as possible inside at least one receive coil.

The at least one characteristic of the patient may be taken from registration information. The registration information may be acquired on registration of the patient before carrying out the magnetic resonance measurement.

Positioning of the patient in the magnetic resonance apparatus may, for example, be configured such that the patient is positioned head first or feet first on the patient couch.

In one embodiment, in each case, one adjustment pilot tone signal is captured (e.g., for fully automatic selection and/or for automatic proposal of the semi-automatic selection of the second set of coil elements) with at least one part of the plurality of coil elements of the magnetic resonance apparatus. The adjustment pilot tone signals are evaluated with regard to respective contribution to ascertaining the patient movement information (e.g., his/her heartbeat and/or his/her respiratory movement). Further, a maximum number K of coil elements of the second set of coil elements is ascertained, where K coil elements are selected and/or proposed that make the biggest contribution to ascertaining the patient movement information.

The maximum number K of coil elements may, for example, correspond to a number L of receive channels that are still available (e.g., for capturing pilot tone signals) after selection of the first set of coil elements (e.g., having received magnetic resonance signals that are used for ascertaining the at least one magnetic resonance image).

The at least one part of the plurality of coil elements of the magnetic resonance apparatus, with which in each case an adjustment pilot tone signal is captured, may be determined with the assistance of one type of at least one receive coil that includes at least one part of the plurality of coil elements of the magnetic resonance apparatus, and/or a relative position of the coil elements within at least one coil that includes at least some coil elements of the plurality of coil elements of the magnetic resonance apparatus, and/or at least one characteristic of the patient and/or a positioning of the patient in the magnetic resonance apparatus (e.g., on the patient couch).

Determination of the at least one part of the plurality of coil elements of the magnetic resonance apparatus may proceed using the same criteria, such as the possible selection of the second set of coil elements, as were described above. In one embodiment, the best coil element candidates are determined with which in each case an adjustment pilot tone signal is to be captured. In this way, it is possible to avoid time-consuming adjustment measurement with all possible coil elements of the magnetic resonance apparatus.

A further embodiment provides that for, for example, semi-automatic and/or manual selection of the coil elements, an avatar of the patient is displayed to an operator, together with at least one part of the plurality of coil elements of the magnetic resonance apparatus correctly positioned in relation to the avatar. It is further displayed whether a displayed coil element is assigned to the first set of coil elements and/or the second set of coil elements.

In one embodiment, with the help of this display, the operator may perform manual selection of the first set of coil elements and the second set of coil elements. Such a selection may proceed particularly clearly and conveniently.

An imaging region may also be displayed to the operator for coil element selection. The imaging region is, for example, a region of the patient, where the magnetic resonance signals generated in this region are received with the coil elements of the first set of coil elements with, for example, the highest possible quality (e.g., the highest possible SNR). In one embodiment, the operator selects coil elements for the first set of coil elements that lie in the imaging region and/or as close as possible to the imaging region.

In one embodiment, those coil elements that lie in the imaging region and/or up to a predetermined distance from the imaging region are automatically proposed to the operator (e.g., in the context of semi-automatic selection). The operator may confirm or change the selection.

Further, in one embodiment, a magnetic resonance apparatus includes a plurality of coil elements that are configured to carry out the above-described embodiments of the method for performing magnetic resonance measurement on a patient.

The magnetic resonance apparatus may have a system control unit for selecting a first set of coil elements from the plurality of coil elements and a second set of coil elements from the plurality of coil elements. The coil elements may be configured to receive magnetic resonance signals and/or pilot tone signals during magnetic resonance measurement.

The system control unit may be configured to ascertain at least one magnetic resonance image solely with the assistance of magnetic resonance signals received with the first set of coil elements during performance of the magnetic resonance measurement. Further, the system control unit may be configured to ascertain patient movement information solely with the assistance of pilot tone signals received solely with the first set of coil elements during performance of the magnetic resonance measurement. The first set of coil elements is not congruent with the second set of coil elements (e.g., there is at least one selected coil element that does not belong to both sets).

The advantages of the magnetic resonance apparatus according to the present embodiments correspond substantially to the advantages of the method according to the present embodiments for performing magnetic resonance measurement on a patient using a magnetic resonance apparatus, which have been described in detail above. Features, advantages, or alternative embodiments mentioned in this connection are likewise also applicable to the other claimed subjects and vice versa.

Further, a computer program product that includes a program and is directly loadable into a memory of a programmable system control unit of a magnetic resonance apparatus and has program means (e.g., libraries and auxiliary functions) for performing a method according to the present embodiments when the computer program product is executed in the system control unit of the magnetic resonance apparatus is provided.

The computer program product may in this respect include software with source code that has yet to be compiled and linked or has merely to be interpreted, or executable software code that has merely to be loaded into the system control unit for execution. As a result of the computer program product, the method according to the present embodiments may be carried out quickly, identically repeatably, and robustly. The computer program product is configured such that the computer program product may carry out the method acts according to the present embodiments by the system control unit. The system control unit in each case includes, for example, the prerequisites such as, for example, an appropriate working memory, an appropriate graphics card, or an appropriate logic unit for it to be possible to carry out the respective method steps efficiently.

The computer program product is, for example, stored on a computer-readable medium (e.g., a non-transitory computer-readable storage medium) or saved to a network or server, from where the computer program product may be loaded into the processor of a local system control unit that may be directly connected with the magnetic resonance apparatus or may be configured as part of the magnetic resonance apparatus. Control information for the computer program product may further be stored on an electronically readable data storage medium (e.g., a non-transitory electronically-readable storage medium). The control information of the electronically readable data storage medium may be configured such that, when the data storage medium is used in a system control unit of a magnetic resonance apparatus, the control information performs a method according to the present embodiments. Examples of electronically readable data storage media are a DVD, a magnetic tape, or a USB stick on which electronically readable control information (e.g., software) is stored. If this control information is read from the data storage medium and stored in a system control unit of the magnetic resonance apparatus, all the embodiments of the previously described methods may be carried out. The present embodiments may accordingly also be based on the computer-readable medium and/or the electronically readable data storage medium.

### BRIEF DESCRIPTION OF THE DRAWINGS

Further advantages, features, and details of the invention are revealed by the exemplary embodiments described below with reference to the drawings. Mutually corresponding parts are provided with the same reference numerals in all the figures.

FIG. 1 is a schematic representation of one embodiment of a magnetic resonance apparatus;

FIG. 2 is a block diagram of one embodiment of a method for performing magnetic resonance measurement; and

FIG. 3 shows one embodiment of a display for the selection of coil elements.

### DETAILED DESCRIPTION

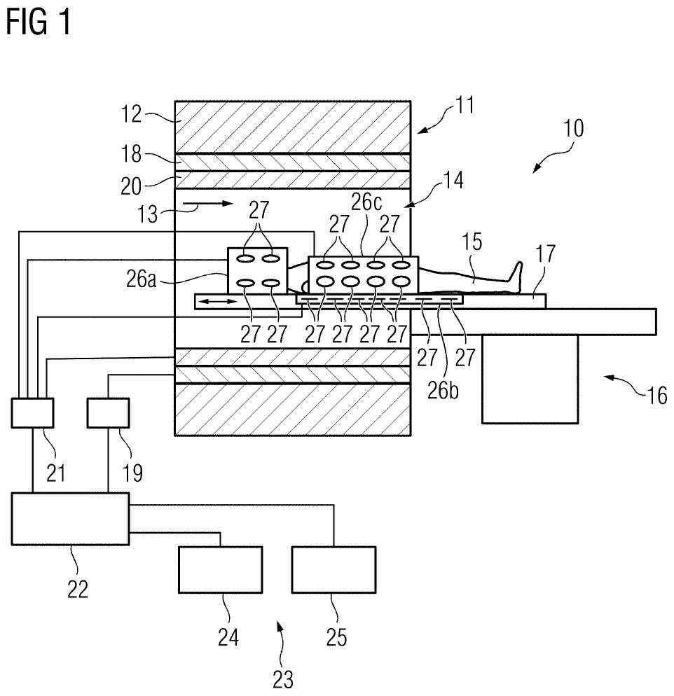

FIG. 1 is a schematic representation of one embodiment of a magnetic resonance apparatus 10. The magnetic resonance apparatus 10 includes a magnet unit 11 that includes a main magnet 12 for generating a strong and, for example, time-constant main magnetic field 13. The magnetic resonance apparatus 10 also includes a patient accommodation zone 14 for accommodating a patient 15. In the present exemplary embodiment, the patient accommodation zone 14 is of cylindrical construction and is cylindrically surrounded in a circumferential direction by the magnet unit 11. In principle, however, the patient accommodation zone 14 may at any time be formed in a manner that differs therefrom. The patient 15 may be advanced into the patient accommodation zone 14 by a patient positioning apparatus 16 of the magnetic resonance apparatus 10. The patient positioning apparatus 16 includes, for example, a patient table 17 that is movable within the patient accommodation zone 14.

The magnet unit 11 also has a gradient coil unit 18 for generating magnetic field gradients that are used for spatial encoding during imaging. The gradient coil unit 18 is controlled by a gradient control unit 19 of the magnetic resonance apparatus 10. The magnet unit 11 further includes a radio-frequency antenna unit 20 that is configured in the present exemplary embodiment as a body coil permanently integrated into the magnetic resonance apparatus 10. The radio-frequency antenna unit 20 is controlled by a radio-frequency antenna control unit 21 of the magnetic resonance apparatus 10 and during magnetic resonance measurement irradiates radio-frequency pulses into an investigation chamber (e.g., into an imaging region FOV) according to a predetermined magnetic resonance sequence. In this way, excitation of atomic nuclei is established in the main magnetic field 13 generated by the main magnet 12. Magnetic resonance signals are generated by relaxation of the excited atomic nuclei. The radio-frequency antenna unit 20 is configured to receive magnetic resonance signals.

The magnetic resonance apparatus 10 includes a system control unit 22 for controlling the main magnet 12 and the gradient control unit 19 and for controlling the radio-frequency antenna control unit 21. The system control unit 22 provides central control of the magnetic resonance apparatus 10, such as, for example, the performance of a predetermined imaging magnetic resonance sequence. In addition, the system control unit 22 includes a system control unit, not shown in greater detail, for evaluating the magnetic resonance signals that are acquired during the magnetic resonance examination. The magnetic resonance apparatus 10 further includes a user interface 23 that is connected to the system control unit 22. Control information, such as, for example, imaging parameters, and reconstructed magnetic resonance images may be displayed on a display unit 24 (e.g., on a screen) of the user interface 23 for a medical operator. The user interface 23 also includes an input unit 25, by which information and/or parameters may be input by the medical operator during a measurement procedure.

The magnetic resonance apparatus further includes three receive coils 23a, 23b, 23c, that are arranged directly on the patient 15. The receive coils 23a, 23b, 23c are thus local coils. Each of the three receive coils 23a, 23b, 23c includes a plurality of coil elements 27 that are conventionally integrated into the respective receive coil 23a, 23b, 23c. The coil elements are capable of receiving RF signals (e.g., magnetic resonance signals and/or pilot tone signals). The received signals may be transmitted to the system control unit 22. With the assistance of the magnetic resonance signals, magnetic resonance images of the patient 15 may be reconstructed by the system control unit 22. With the assistance of the pilot tone signals, information may, for example, be ascertained by the system control unit 22 about movements of the patient 15. The receive coil 23c, for example, includes a pilot tone signal generator (not shown here) that emits an RF signal. This RF signal enters into interaction with the patient 15. For example, the RF signal is influenced by movement of the patient 15. For example, the RF signal may have a different amplitude and/or phase depending on the movement state of the patient 15. The RF signal is received as a pilot tone signal by the coil elements 27 and forwarded to the system control unit 22 for evaluation (e.g., for ascertaining movement information).

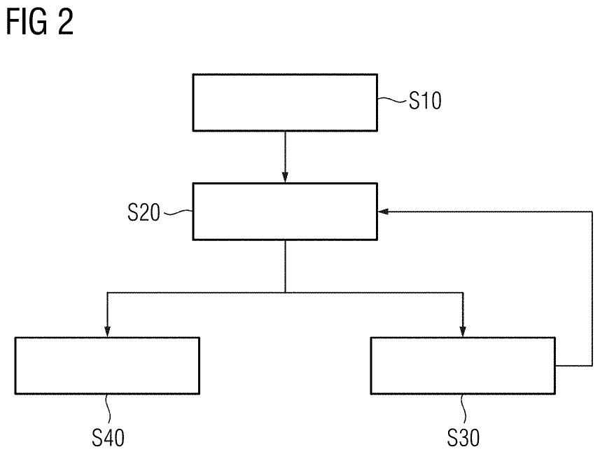

FIG. 2 shows one possible method for performing magnetic resonance measurement on the patient 15 using the magnetic resonance apparatus 10.

In S10, a first set A and a second set B of coil elements are selected from the plurality of coil elements 27. The first set A is in this case not congruent with the second set B (e.g., A≠B). Selection may proceed fully automatically, semi-automatically, and/or manually (e.g., with the assistance of the system control unit 22).

In S20, magnetic resonance measurement is performed, where, during the magnetic resonance measurement with the first set and the second set of coil elements (e.g., with the union of the first and second sets A∪B), magnetic resonance signals and pilot tone signals are received.

In S30, patient 15 movement information is determined solely with the assistance of pilot tone signals received in S20 with the second set of coil elements B during performance of the magnetic resonance measurement. Any pilot tone signals that were received with coil elements of the difference A\B are thus not used for ascertaining the movement information.

The movement information may, for example, be ascertained during the magnetic resonance measurement. The ascertained movement information may, for example, be used for control of the magnetic resonance measurement (e.g., for a prospective movement correction). In this case, a magnetic resonance sequence underlying the magnetic resonance measurement may be adapted in real time and/or on-the-fly (e.g., by the system control unit 22 adapting a position of a slice to be captured).

In step S40, at least one magnetic resonance image of the patient 15 is determined solely with the assistance of magnetic resonance signals received in S20 with the second set of coil elements B during performance of the magnetic resonance measurement. Any pilot tone signals that were received with coil elements of the difference B\A are thus not used for ascertaining the least one magnetic resonance image.

Possible embodiments of the selection of the first set A and second set B are explained in greater detail below. According to a first embodiment, first, the first set A is selected, which is to be used for imaging, prior to selection of the second set B. The first set A may, for example, be performed manually by the operator of the magnetic resonance apparatus 10 or proceed automatically.

In this case, N coil elements are selected from the plurality of coil elements, for example. In one exemplary magnetic resonance apparatus with M receive channels, L=M−N channels remain as receive channels for other applications than imaging, such as, for example, for receiving dedicated pilot tone signals.

Selection of the second set B may, for example, proceed fully automatically (e.g., in a “concealed” manner, unnoticed by the operator). Selection of the second set B and ascertainment of the movement information may, for example, be triggered by the operator activating respiratory monitoring or triggering (e.g., in the context of adjustment of the magnetic resonance measurement).

The magnetic resonance apparatus may automatically detect which of coil elements 27 are particularly favorable for respiratory monitoring. In an upstream adjusting step, adjustment data (e.g., adjustment pilot tone signals) may be captured by potentially favorable receive coils.

Potentially favorable receive coils are, for example, coils that are arranged in the region of the abdomen and/or thorax of the patient. Typically, these are dedicated receive coils, such that selection of the second set B may also proceed with the assistance of the type of at least one receive coil.

In one embodiment, coil elements of specific coil types that lie and are used in the abdomen/thorax region (e.g., a spinal column coil and/or a body matrix coil) are selected.

In one embodiment, a relative position of the receive coils 26a, 26b, 26c (e.g., the coil elements 27 of the receive coils 26a, 26b, 26c) is ascertained. In this case, coil elements lying on the inside in the right-left direction are more important than those on the outside. Selection of the second set B may thus, for example, proceed from a relative position of the coil elements within at least one receive coil.

For example, prior information obtained from registration of the patient 15 may be used to ascertain where the coil elements with the highest signal contributions may probably be located. Such prior information may, for example, include positioning (e.g., planned positioning) of the patient 15 in the magnetic resonance apparatus 10 (e.g., whether the patient is introduced into the patient accommodation zone 14 head first or feet first). Further, the prior information may, for example, include at least one characteristic of the patient 15 (e.g., their height, weight, and/or sex).

If the magnetic resonance apparatus has a sufficient number of receive channels, adjustment data may be captured from all the coil elements (e.g., relevant coil elements). If not, a preselection of the above-described information and/or criteria may be put together. The elements may also be captured in a number of groups one after the other or in temporally interleaved manner.

The adjustment data is examined (e.g., with a principal component analysis) with regard to how great is the contribution of the individual coil elements to detection of the movement information (e.g., a respiratory signal). In this way, an order of precedence of the particularly suitable coil elements may be drawn up.

The first L coil elements from this selection may, for example, be selected by the magnetic resonance apparatus 10 for receiving pilot tone signals, without the first set A, including the coil elements for the imaging, being limited. In addition to the first L coil elements, the second set B may, for example, also include suitable coil elements from the first set A. For example, a maximum number K of coil elements of the second set B may be ascertained, such that in addition to the first L coil elements, still further K−L coil elements from the first set A may additionally be selected.

If L is very small or zero or no signal has been found that is sufficiently strong for a movement (e.g., respiration), it may, for example, be displayed to the operator that pilot tone-based monitoring has been deactivated.

A coil element used in magnetic resonance measurement in S20 may thus belong only to set A, only to set B, or to both sets. For example, information is transmitted to the hardware of the magnetic resonance apparatus 10, for performance of the magnetic resonance measurement, about which coil elements are selected (e.g., the union A∪B of sets A and B). The software of the magnetic resonance apparatus 10 may, for example, distinguish to which end a coil element was selected (e.g., whether a coil element belongs only to set A, only to set B, or to set A∪B).

For example, the display unit 24 may display to the operator only the coil elements of set A for imaging, but not the coil elements of set B for movement detection.

According to one further embodiment, selection of the second set B proceeds manually by the operator. To this end, possible coil elements are displayed (e.g., via the display unit 24) to the operator, who may assign the possible coil elements the second set B, for example, by clicking on the possible coil elements. This selection option may be configured such that it is clear to the operator that, with the received signals of the coil elements to be selected, no image reconstruction is to proceed; rather, the received signals merely serve to ascertain the movement information with the assistance of the pilot tone signals received thereby.

According to one further embodiment, the second set B of coil elements is fully automatically selected (e.g., on activation of pilot tone functionality). For example, the fully automatically selected coil elements cannot be deselected again by the operator. The operator may then, insofar as permitted by the number of available receive channels of the magnetic resonance apparatus, manually select additional coil elements for imaging. Alternatively, the coil elements are fully automatically selected for imaging (e.g., based on imaging geometry). Selection of the second set of coil elements thus proceeds, for example, prior to selection of the first set of coil elements.

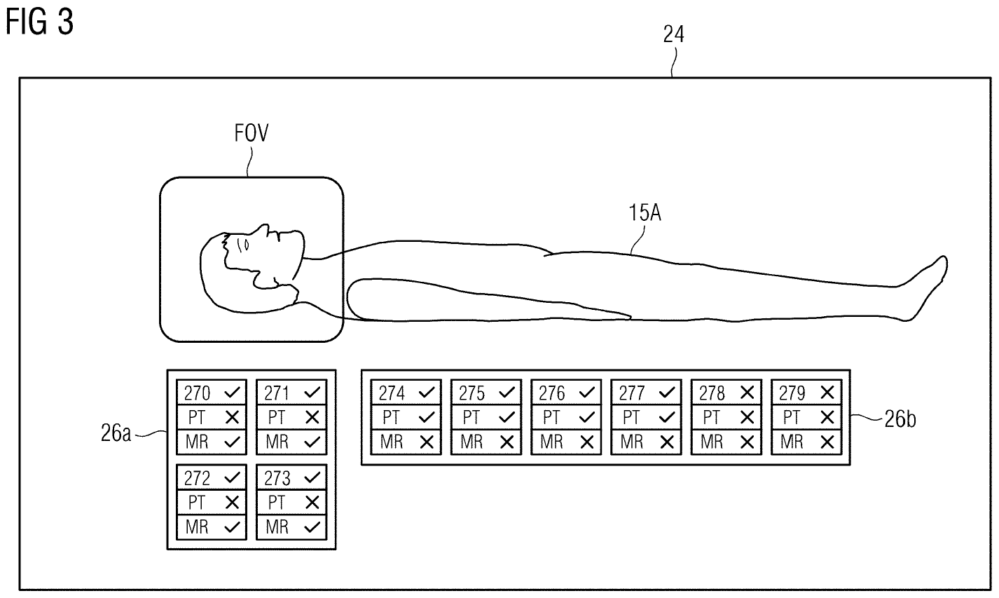

FIG. 3 shows a representation, by way of example, that is displayed to the operator by the display unit 24 on selection of the coil elements. In this case, a patient avatar 15A and receive coils 26a, 26b, positioned correctly relative thereto, with their coil elements 270-279 are displayed, which may be used during the following magnetic resonance measurement. The receive coil 26a, which is, for example, a head coil, includes the coil elements 270, 271, 272, 273; the receive coil 26a, which is, for example, a spinal column coil, includes the coil elements 274, 275, 276, 277, 278, 279. The position of the receive coils 26a, 26b or their coil elements 270-279 may, for example, be ascertained by an adjustment measurement that precedes the actual magnetic resonance measurement and during which magnetic resonance signals are conventionally evaluated to determine the position. Further, in the case of stationary receive coils, such as, for example, typically a head coil, the positions of the coil elements may be stored in a coil-specific file. Further, to determine the position of the receive coils and/or of the coil elements, 2D and/or 3D cameras, for example, and/or sensors attached to the receive coil (e.g., Hall sensors) may be used.

It is further displayed whether a displayed coil element is assigned to the first set of coil elements and/or the second set of coil elements. For example, it is depicted which coil elements belong to the second set B (e.g., are provided for ascertaining movement information), and which coils elements belong to the first set A (e.g., are provided for imaging). If a coil element belongs to the first set A, this is represented by “MR” in conjunction with a check mark; if a coil element belongs to the second set B, this is represented by “PT” in conjunction with a check mark. If a coil element does not belong to the first set A, this is represented by “MR” in conjunction with a cross; if a coil element does not belong to the second set B, this is represented by “PT” in conjunction with a cross. If a coil element belongs to the union A∪B (e.g., is to receive any RF signals), this is represented by its coil number in conjunction with a check mark; otherwise, this is represented by a cross. In the case shown, coil elements 270, 271, 272, 273 belong to the first set A; the magnetic resonance signals received thereby are particularly well suited to ascertaining therefrom at least one magnetic resonance image of the patient 15. Coil elements 274, 275, 276, 277 belong to the second set B; the pilot tone signals received thereby are particularly well suited to detecting therefrom the movement of the heart and/or the abdomen of the patient 15. Coil elements 278, 279, in contrast, are not used in magnetic resonance measurement.

Selection of the coil elements may, for example, proceed with the assistance of one characteristic of the displayed coil element (e.g., its position): if the display is then supplemented by the depiction of an imaging region FOV (e.g., an imaging volume), it immediately becomes clear which coil element is suitable for which purpose. Above all, the coil elements that are located within the imaging region FOV or close to the imaging region FOV contribute to the image. The criterion of the position of the receive coils relative to the imaging region FOV may also lead, after performance of the magnetic resonance measurement, to the removal of coil elements from the first set A (e.g., the magnetic resonance signals that were received with such remote coil elements are then not used to ascertain the at least one magnetic resonance image).

In one embodiment, the coil elements are selected with the assistance of geometric criteria. Additionally or alternatively, the coil symbols outside the imaging region FOV may also be modified to display that the coil symbols do not represent imaging coil elements of set A. The modification may be limited to an interface with the operator; in other parts of the magnetic resonance apparatus 10, the two coil types would not advantageously need to be distinguished.

To summarize, it may be noted that the method of the present embodiments makes pilot tone-based monitoring possible also in areas of the body in which coil elements are not used for imaging (e.g., when measuring the head of the patient 15 with heartbeat and/or respiratory synchronization). In one embodiment, the operator does not herself need to select such coil elements.

The method described above in detail and the depicted magnetic resonance apparatus are merely exemplary embodiments that may be modified in the most varied manner by a person skilled in the art without departing from the scope of the invention. Further, use of the indefinite article “a” does not rule out the possibility of a plurality of the features in question also being present. Likewise, the term “unit” does not rule out the possibility of the components in question consisting of a plurality of interacting subcomponents that may optionally also be spatially distributed.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for performing a magnetic resonance measurement of a patient using a magnetic resonance apparatus comprising a plurality of coil elements for receiving RF signals, the method comprising:
selecting a first set of coil elements from the plurality of coil elements and a second set of coil elements from the plurality of coil elements;
performing the magnetic resonance measurement, wherein magnetic resonance signals and pilot tone signals are received during the magnetic resonance measurement with the first set of coil elements and the second set of coil elements;
ascertaining at least one magnetic resonance image solely with assistance of magnetic resonance signals received with the first set of coil elements during performance of the magnetic resonance measurement; and
ascertaining patient movement information solely with assistance of pilot tone signals received with the second set of coil elements during performance of the magnetic resonance measurement,
wherein the first set of coil elements is not congruent with the second set of coil elements.

2. The method of claim 1, wherein the selecting of the first set of coil elements proceeds prior to the selecting of the second set of coil elements.

3. The method of claim 1, wherein the selecting of the second set of coil elements proceeds prior to the selecting of the first set of coil elements.

4. The method of claim 1, wherein the first set of coil elements and the second set of coil elements are jointly selected.

5. The method of claim 4, wherein a weighting factor that describes a relative prioritization of a probable quality of the at least one magnetic resonance image to be ascertained compared with a quality of the patient movement information to be ascertained is defined, and
wherein the joint selection of the first set of coil elements and the second set of coil elements proceeds with assistance of the weighting factor.

6. The method of claim 1, further comprising ascertaining, at least for a part of the plurality of coil elements of the magnetic resonance apparatus, a position of the part of the plurality of coil elements relative to the magnetic resonance apparatus, relative to the patient, or relative to the magnetic resonance apparatus and relative to the patient, and
wherein selecting the first set of coil elements, the second set of coil elements, or the first set of coil elements and the second set of coil elements in each case proceeds as a function of the respectively ascertained position.

7. The method of claim 6, wherein the positions of the coil elements are ascertained using:
evaluation of a stored item of coil-specific information that describes a position of at least one coil element relative to the receive coil, which comprises the at least one coil element;
evaluation of magnetic resonance signals that were received during an adjustment measurement with the coil elements;
evaluation of a video signal that was captured with at least one camera;
evaluation of a sensor signal that was acquired with at least one position sensor; or
any combination thereof.

8. The method of claim 1, wherein selecting the first set of coil elements, the second set of coil elements, or the first set of coil elements and the second set of coil elements takes place fully automatically, semi-automatically, manually, or any combination thereof,
wherein, in the case of fully automatic selecting of coil elements, an operator of the magnetic resonance apparatus plays no part in selection of the coil elements, and
wherein, in the case of semi-automatic selecting of coil elements, a set of coil elements is automatically proposed to the operator, and the coil elements are selectable by the operator based on the proposal.

9. The of claim 1, wherein selecting the second set of coil elements proceeds with the assistance of:
one type of at least one receive coil that comprises at least one part of the plurality of coil elements of the magnetic resonance apparatus;
a relative position of the coil elements within at least one receive coil that comprises at least one part of the plurality of coil elements of the magnetic resonance apparatus;
at least one characteristic of the patient;
positioning of the patient in the magnetic resonance apparatus; or
any combination thereof.

10. The method of claim 1, wherein in each case one adjustment pilot tone signal is captured with at least one part of the plurality of coil elements of the magnetic resonance apparatus,
wherein the adjustment pilot tone signals are evaluated in terms of respective contribution to ascertaining the patient movement information,
wherein a maximum number K of coil elements of the second set of coil elements is ascertained, and
wherein K coil elements that make a biggest contribution to ascertaining the patient movement information are selected, proposed, or selected and proposed.

11. The method of claim 10, wherein the at least one part of the plurality of coil elements of the magnetic resonance apparatus, with which in each case an adjustment pilot tone signal is captured, is determined with the assistance of:
one type of at least one receive coil that comprises at least one part of the plurality of coil elements of the magnetic resonance apparatus;
a relative position of the coil elements within at least one coil that comprises at least one part of the plurality of coil elements of the magnetic resonance apparatus;
at least one characteristic of the patient;
positioning of the patient in the magnetic resonance apparatus; or
a combination thereof.

12. The method of claim 1, further comprising:
for selecting the first set of coil elements from the plurality of coil elements and the second set of coil elements from the plurality of coil elements, displaying an avatar of the patient to an operator, together with at least one part of the plurality of coil elements of the magnetic resonance apparatus correctly positioned in relation to the avatar; and
displaying whether a displayed coil element is assigned to the first set of coil elements, the second set of coil elements, or the first set of coil elements and the second set of coil elements.

13. The method of claim 12, further comprising displaying an imaging region to the operator for coil element selection.

14. A magnetic resonance apparatus for performing a magnetic resonance measurement of a patient, the magnetic resonance apparatus comprising:
a processor; and
a plurality of coil elements for receiving RF signals,
wherein the processor is configured to select a first set of coil elements from the plurality of coil elements and a second set of coil elements from the plurality of coil elements,
wherein the plurality of coil elements are configured to perform the magnetic resonance measurement, wherein the first set of coil elements and the second set of coil elements are configured to receive magnetic resonance signals and pilot tone signals during the magnetic resonance measurement,
wherein the processor is further configured to:
ascertain at least one magnetic resonance image solely with assistance of magnetic resonance signals received with the first set of coil elements during performance of the magnetic resonance measurement; and
ascertain patient movement information solely with assistance of pilot tone signals received with the second set of coil elements during performance of the magnetic resonance measurement, and
wherein the first set of coil elements is not congruent with the second set of coil elements.

15. In a non-transitory computer-readable storage medium that stores instructions executable by one or more processors to perform a magnetic resonance measurement of a patient using a magnetic resonance apparatus comprising a plurality of coil elements for receiving RF signals, the instructions comprising:
selecting a first set of coil elements from the plurality of coil elements and a second set of coil elements from the plurality of coil elements;
performing a magnetic resonance measurement, wherein magnetic resonance signals and pilot tone signals are received during the magnetic resonance measurement with the first set of coil elements and the second set of coil elements;
ascertaining at least one magnetic resonance image solely with assistance of magnetic resonance signals received with the first set of coil elements during performance of the magnetic resonance measurement; and
ascertaining patient movement information solely with assistance of pilot tone signals received with the second set of coil elements during performance of the magnetic resonance measurement,
wherein the first set of coil elements is not congruent with the second set of coil elements

16. The non-transitory computer-readable storage medium of claim 15, wherein the selecting of the first set of coil elements proceeds prior to the selecting of the second set of coil elements.

17. The non-transitory computer-readable storage medium of claim 15, wherein the selecting of the second set of coil elements proceeds prior to the selecting of the first set of coil elements.

18. The non-transitory computer-readable storage medium of claim 15, wherein the first set of coil elements and the second set of coil elements are jointly selected.

19. The non-transitory computer-readable storage medium of claim 18, wherein a weighting factor that describes a relative prioritization of a probable quality of the at least one magnetic resonance image to be ascertained compared with a quality of the patient movement information to be ascertained is defined, and
wherein the joint selection of the first set of coil elements and the second set of coil elements proceeds with assistance of the weighting factor.
